(19) United States
(12) Patent Application Publication
(10) Pub. No.: US 2022/0095939 A1
(43) Pub. Date: Mar. 31, 2022

(54) BLOOD PRESSURE MONITORING DEVICE

(71) Applicant: Trey Cooke, Beebe, AR (US)

(72) Inventor: Trey Cooke, Beebe, AR (US)

(21) Appl. No.: 17/034,511

(22) Filed: Sep. 28, 2020

(51) Int. Cl. A61B 5/021; A61B 17/132; A61B 5/00; A61B 5/1455

(52) U.S. Cl. CPC A61B 5/02141; A61B 17/1322; A61B 5/6813; A61B 5/14551; A61B 5/6831

(57) ABSTRACT

A blood pressure monitoring device, including a cuff assembly to connect around a limb of a user, a blood pressure monitor assembly disposed at a first side on at least a portion of the cuff assembly to detect at least one of a blood pressure level and a heart rate of the user, and a tourniquet assembly disposed on at least a portion of a second side of the blood pressure monitor assembly to at least partially decrease a diameter the tourniquet assembly around the limb of the user in a first position and at least partially increase the diameter of the tourniquet assembly around the limb of the user in a second position.

### BACKGROUND

### 1. Field

The present general inventive concept relates generally to a health monitoring device, and particularly, to a blood pressure monitoring device.

### 2. Description of the Related Art

First responders are important workers in human society because they are usually the first people to arrive at the scene of an emergency, such as an accident, a fire, and/or an injury sustained by people and/or animals. A first responder usually includes law enforcement officers, firefighters, paramedics, and emergency medical technicians (EMTs).

An emergency causes a great deal of pressure to the first responders and other medical practitioners because it is not always immediately known what type of support is necessary. In the case of a physical injury, the paramedics, the EMTs, and/or the medical practitioner will measure blood pressure of an injured person, but could also require treating an injury that causes blood loss. Unfortunately, this requires having the appropriate equipment, which may not be available.

Therefore, there is a need for a blood pressure monitoring device that can be used for multiple medical purposes.

### SUMMARY

The present general inventive concept provides a blood pressure monitoring device.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a blood pressure monitoring device, including a cuff assembly to connect around a limb of a user, a blood pressure monitor assembly disposed at a first side on at least a portion of the cuff assembly to detect at least one of a blood pressure level and a heart rate of the user, and a tourniquet assembly disposed on at least a portion of a second side of the blood pressure monitor assembly to at least partially decrease a diameter the tourniquet assembly around the limb of the user in a first position and at least partially increase the diameter of the tourniquet assembly around the limb of the user in a second position.

The tourniquet assembly may include a tourniquet layer, a strap receiving channel disposed within at least a portion of the tourniquet layer, and a strap disposed within the strap receiving channel to connect around the limb of the user.

The strap may include a plurality of teeth disposed on at least a portion of a first edge and a second edge of the strap to protrude away from the first edge and the second edge of the strap.

The tourniquet assembly may further include a plurality of gears disposed within at least a portion of the strap receiving channel to connect to the plurality of teeth to move the strap within the strap receiving channel in a first rotational direction and away from the strap receiving channel in a second rotational direction, and a motor disposed within at least a portion of the strap receiving channel and connected to the plurality of gears to rotate the plurality of gears in the first rotational direction or the second rotational direction in response to rotation of the motor.

The blood pressure monitor assembly may be disposed between the cuff assembly and the tourniquet assembly.

The blood pressure monitoring device may further include a pulse oximeter disposed within at least a portion of the cuff assembly to monitor an oxygen saturation level of the user.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1 illustrates a top perspective view of a blood pressure monitoring device, according to an exemplary embodiment of the present general inventive concept; and

FIG. 2 illustrates a zoomed in view of a tourniquet assembly, according to an exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

### LIST OF COMPONENTS

Blood Pressure Monitoring Device 100
Cuff Assembly 110
Outer Layer 111
Fastener 112
Fastener Receiver 113
Blood Pressure Monitor Assembly 120
Blood Pressure Monitor Layer 121
Air Pump 122
Solenoid Valve 123
Sensor 124
Tourniquet Assembly 130
Tourniquet Layer 131
Gauze Receiving Portion 131a
Strap 132
Teeth 132a
Strap Fastener 132b
Strap Receiving Channel 133
Gears 134
Motor 135
Display Unit 140
Control Unit 150
Power Button 151
Blood Pressure Button 152
Tourniquet Button 153
Pulse Oximeter 160
Power Source 170

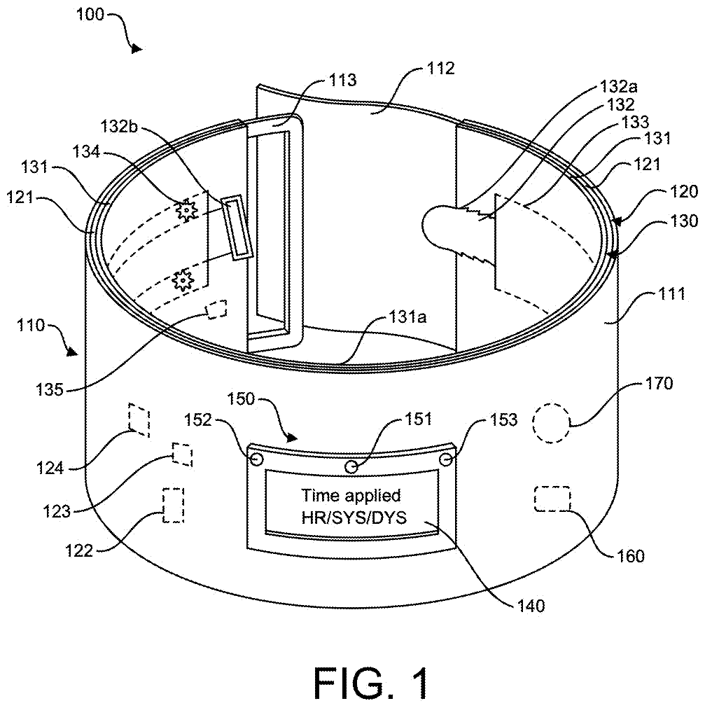

FIG. 1 illustrates a top perspective view of a blood pressure monitoring device 100, according to an exemplary embodiment of the present general inventive concept.

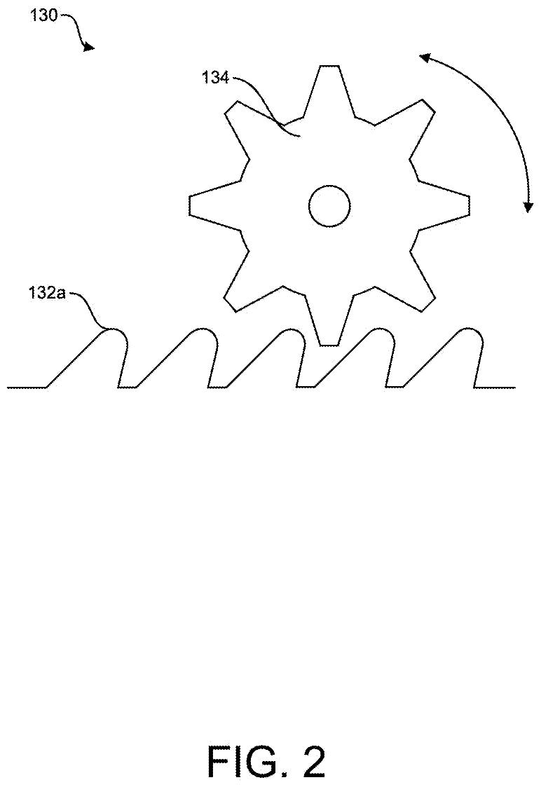

FIG. 2 illustrates a zoomed in view of a tourniquet assembly 130, according to an exemplary embodiment of the present general inventive concept.

The blood pressure monitoring device 100 may be constructed from at least one of metal, plastic, cloth, and rubber, etc., but is not limited thereto. Also, the blood pressure monitoring device may be sterile, reusable medical grade equipment.

The blood pressure monitoring device 100 may include a cuff assembly 110, a blood pressure monitor assembly 120, a tourniquet assembly 130, a display unit 140, a control unit 150, a pulse oximeter 160, and a power source 170, but is not limited thereto.

The cuff assembly 110, the blood pressure monitor assembly 120, and/or the tourniquet assembly 130 may have a predetermined size, including a length and a width, to connect and/or fit (i.e. wrap) around a limb, such as an arm, a wrist, a leg, and/or an ankle of a user.

The cuff assembly 110 may include an outer layer 111, a fastener 112, and a fastener receiver 113, but is not limited thereto.

The outer layer 111 may be constructed to be highly durable and flexible, such that the outer layer 111 may at least partially deform in a first direction or a second direction opposite to the first direction with respect to at least one plane (i.e. a first plane and/or a second plane).

The fastener 112 may include hooks and loops, a buckle, a snap, a clasp, a magnet, a zipper, a button, a tape, and a glue, but is not limited thereto.

The fastener 112 may be disposed on at least a portion of a first end of the outer layer 111.

Referring to FIG. 1, the fastener receiver 113 is illustrated to be a loop. However, the fastener receiver may include hooks and loops, a buckle, a snap, a clasp, a magnet, a zipper, a button, a tape, and a glue, but is not limited thereto.

The fastener receiver 113 may be disposed on at least a portion of a second end of the outer layer 111. The fastener 112 may connect to the fastener receiver 113 to close the outer layer 111 around the limb of the user. In other words, the fastener receiver 113 may receive and connect to the fastener 112 thereupon. Alternatively, the fastener receiver 113 may receive the fastener 112 through the loop, such that the fastener 112 may connect through the loop to another hooks and loops of the fastener receiver 113 disposed on at least a portion of the second end of the outer layer 111.

The blood pressure monitor assembly 120 may include a blood pressure monitor layer 121, an air pump 122, a solenoid valve 123, and a sensor 124, but is not limited thereto.

The blood pressure monitor layer 121 may be constructed to be highly durable and flexible, such that the blood pressure monitor layer 121 may at least partially deform in the first direction or the second direction opposite to the first direction with respect to the at least one plane. Also, a first side of the blood pressure monitor layer 121 may be disposed on at least a portion of the outer layer 111.

The air pump 122 may be disposed within at least a portion of the blood pressure monitor layer 121. Moreover, the air pump 122 may move air from an exterior portion of the blood pressure monitor layer 121 to an interior portion of the blood pressure monitor layer 121, such that the blood pressure monitor layer 121 may inflate with the air.

The solenoid valve 123 may be disposed within at least a portion of the blood pressure monitor layer 121. The solenoid valve 123 may be connected to the air pump 121 to allow and/or prevent movement of the air from the air pump 122 and/or the blood pressure monitor layer 121.

The sensor 124 may be disposed within at least a portion of the blood pressure monitor layer 121. The sensor 124 may be electrically connected to the air pump 121 and/or the solenoid valve 123. Additionally, the sensor 124 may detect a blood pressure level and/or a heart rate of the user. More specifically, the sensor 124 may allow the air pump 122 using the solenoid valve 123 to move air into the blood pressure monitor layer 121 in response to activation thereof. Subsequently, the sensor 124 may allow the air pump 122 using the solenoid valve 123 to extract the air from the blood pressure monitor layer 121 in response to detecting an absence of the blood pressure level during movement of the air within the blood pressure monitor layer 121.

During extraction of the air from the blood pressure monitor layer 121, the sensor 124 may determine the blood pressure level and/or the heart rate of the user.

The tourniquet assembly 130 may include a tourniquet layer 131, a strap 132, a strap receiving channel 133, a plurality of gears 134, and a motor 135, but is not limited thereto.

The tourniquet layer 131 may be constructed to be highly durable and flexible, such that the tourniquet layer 131 may at least partially deform in the first direction or the second direction opposite to the first direction with respect to the at least one plane. Also, the tourniquet layer 131 may be disposed on at least a portion of a second side of the blood pressure monitor layer 121. As such, the blood pressure monitor layer 121 may be disposed between the outer layer 111 and/or the tourniquet layer 131. Accordingly, the tourniquet layer 131 may be separate from the blood pressure monitor layer 121 to prevent interference with operation of the blood pressure monitor layer 121.

The tourniquet layer 131 may include a gauze receiving portion 131a, but is not limited thereto.

The gauze receiving portion 131a may be receive at least one piece of gauze thereon. Moreover, the gauze receiving portion 131a may be recessed with respect to a surface of the tourniquet layer 131.

The strap 132 and/or the strap receiving channel 133 may be disposed within at least a portion of the tourniquet layer 131. Also, a length of the strap receiving channel 133 may extend a length of the tourniquet layer 131. Furthermore, the strap 132 may be disposed within at least a portion of the strap receiving channel 133. The strap 132 may move in a first lateral direction or a second lateral direction through the strap receiving channel 133.

The strap 132 may include a plurality of teeth 132a and a strap fastener 132b, but is not limited thereto.

The plurality of teeth 132a may be disposed at a first end of the strap 132 on at least a portion of a first edge of the strap 132 and a second edge of the strap 132 opposite with respect to the first edge. Moreover, the plurality of teeth 132 may be disposed an entire length of the strap 132. The plurality of teeth 132a may protrude away from the first edge of the strap 132 and the second edge of the strap 132.

The strap fastener 132b may include hooks and loops, a buckle, a snap, a clasp, a magnet, a zipper, a button, a tape, and a glue, but is not limited thereto.

The strap fastener 132b may be disposed on a second end of the strap 132. The strap fastener 132b may receive and connect to the plurality of teeth 132a. As such, the strap 132 may connect around the limb of the user in response to connecting the plurality of teeth 132a to the strap fastener 132b.

The plurality of gears 134 may be disposed within at least a portion of the strap receiving channel 133. More specifically, the plurality of gears 134 may connect to the plurality of teeth 132a. Also, the plurality of gears 134 may rotate in response to activation thereof.

The motor 135 may be disposed within at least a portion of the strap receiving channel 133 and/or the tourniquet layer 131. Additionally, the motor 135 may be connected to the plurality of gears 134, such that the plurality of gears may rotate in response to rotation of the motor 135.

The display unit 140 may include a plasma screen, an LCD screen, a light emitting diode (LED) screen, an organic LED (OLED) screen, a computer monitor, a hologram output unit, a sound outputting unit, or any other type of device that visually or aurally displays data.

The display unit 140 may be disposed on at least a portion of the outer layer 111. Moreover, the display unit 140 may display the blood pressure level and/or the heart rate of the user thereon based on a reading from the sensor 124. Specifically, the display unit 140 may display a systolic pressure, a diastolic pressure, and/or the heart rate in beats per minute (bpm).

The control unit 150 may include a power button 151, a blood pressure button 152, and a tourniquet button 153, but is not limited thereto.

The control unit 150 may be disposed on at least a portion of the display unit 140. The power button 151 may turn on and/or turn off the blood pressure monitor assembly 120, the tourniquet assembly 130, the display unit 140, and/or the control unit 150 in response to being depressed a first time and a second time, respectively.

The blood pressure monitor assembly 120 may operate in response to depressing the blood pressure button 152. In other words, the air pump 122 may move the air into the blood pressure monitor layer 121 in response to depressing the blood pressure button 152.

The tourniquet assembly 130 may operate in response to depressing the tourniquet button 153. In other words, the motor 135 may rotate in a first rotational direction in response to depressing the tourniquet button 153 a first time, such that the plurality of gears 134 may rotate in the first rotational direction against the plurality of teeth 132a to move the strap 132 in the first lateral direction or the second lateral direction within the second end of the tourniquet layer 131 and/or the strap receiving channel 133. The motor 135 may stop rotating in response to depressing the tourniquet button 153 a second time. Alternatively, the motor may rotate in a second rotational direction in response to depressing the tourniquet button 153 a third time, such that the plurality of gears 134 may rotate in the second rotational direction against the plurality of teeth 132 to move the strap 132 in the second lateral direction or the first lateral direction away from the second end of the tourniquet layer 131 and/or the strap receiving channel 133.

Also, the sensor 124 may connect to the motor 135 to detect a pressure level compared to a predetermined pressure level based on a percent reduction in the blood pressure level and/or the heart rate of the user, such that the motor 135 may stop rotating in response to the pressure level exceeding the predetermined pressure level.

Therefore, the strap 132 may at least partially constrict (i.e. decrease a diameter of) the tourniquet layer 131 around the limb of the user in a first position in response to rotation of the motor 135 in the first rotational direction to rotate the plurality of gears 134 in the first rotational direction and at least partially expand (i.e. increase a diameter of) the tourniquet layer 131 around the limb of the user in a second position in response to rotation of the motor 135 in the second rotational direction to rotate the plurality of gears 134 in the second rotational direction.

The pulse oximeter 160 may be disposed within at least a portion of the outer layer 111, the blood pressure monitor layer 121, and/or the tourniquet layer 131. The pulse oximeter 160 may monitor an oxygen saturation level in the user. As such, the display unit 140 may display the oxygen saturation level thereon.

The power source 170 may include a battery and a solar cell, but is not limited thereto.

The power source 170 may be disposed within at least a portion of the outer layer 111, the blood pressure monitor layer 121, and/or the tourniquet layer 131. The power source 170 may provide power to the blood pressure monitor assembly 120, the tourniquet assembly 130, the display unit 140, the control unit 150, and/or the pulse oximeter 160.

Therefore, the blood pressure monitoring device 100 may allow a first responder to quickly aid the user. Furthermore, the blood pressure monitoring device 100 may provide a blood pressure reading, a tourniquet, and/or a pulse oximeter reading.

The present general inventive concept may include a blood pressure monitoring device 100, including a cuff assembly 110 to connect around a limb of a user, a blood pressure monitor assembly 120 disposed at a first side on at least a portion of the cuff assembly 110 to detect at least one of a blood pressure level and a heart rate of the user, and a tourniquet assembly 130 disposed on at least a portion of a second side of the blood pressure monitor assembly 120 to at least partially decrease a diameter the tourniquet assembly 130 around the limb of the user in a first position and at least partially increase the diameter of the tourniquet assembly 130 around the limb of the user in a second position.

The tourniquet assembly 130 may include a tourniquet layer 131, a strap receiving channel 133 disposed within at least a portion of the tourniquet layer 131, and a strap 132 disposed within the strap receiving channel 133 to connect around the limb of the user.

The strap 132 may include a plurality of teeth 132a disposed on at least a portion of a first edge and a second edge of the strap 132 to protrude away from the first edge and the second edge of the strap 132.

The tourniquet assembly 130 may further include a plurality of gears 134 disposed within at least a portion of the strap receiving channel 133 to connect to the plurality of teeth 132a to move the strap within the strap receiving channel 133 in a first rotational direction and away from the strap receiving channel 133 in a second rotational direction, and a motor 135 disposed within at least a portion of the strap receiving channel 133 and connected to the plurality of gears 134 to rotate the plurality of gears 134 in the first rotational direction or the second rotational direction in response to rotation of the motor 135.

The blood pressure monitor assembly 120 may be disposed between the cuff assembly 110 and the tourniquet assembly 130.

The blood pressure monitoring device 100 may further include a pulse oximeter 160 disposed within at least a portion of the cuff assembly 110 to monitor an oxygen saturation level of the user.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A blood pressure monitoring device, comprising:
a cuff assembly to connect around a limb of a user;
a blood pressure monitor assembly disposed at a first side on at least a portion of the cuff assembly to detect at least one of a blood pressure level and a heart rate of the user; and
a tourniquet assembly disposed on at least a portion of a second side of the blood pressure monitor assembly to at least partially decrease a diameter the tourniquet assembly around the limb of the user in a first position and at least partially increase the diameter of the tourniquet assembly around the limb of the user in a second position.

2. The blood pressure monitoring device of claim 1, wherein the tourniquet assembly comprises:
a tourniquet layer;
a strap receiving channel disposed within at least a portion of the tourniquet layer; and
a strap disposed within the strap receiving channel to connect around the limb of the user.

3. The blood pressure monitoring device of claim 2, wherein the strap comprises:
a plurality of teeth disposed on at least a portion of a first edge and a second edge of the strap to protrude away from the first edge and the second edge of the strap.

4. The blood pressure monitoring device of claim 3, wherein the tourniquet assembly further comprises:
a plurality of gears disposed within at least a portion of the strap receiving channel to connect to the plurality of teeth to move the strap within the strap receiving channel in a first rotational direction and away from the strap receiving channel in a second rotational direction; and
a motor disposed within at least a portion of the strap receiving channel and connected to the plurality of gears to rotate the plurality of gears in the first rotational direction or the second rotational direction in response to rotation of the motor.

5. The blood pressure monitoring device of claim 1, wherein the blood pressure monitor assembly is disposed between the cuff assembly and the tourniquet assembly.

6. The blood pressure monitoring device of claim 1, further comprising:
a pulse oximeter disposed within at least a portion of the cuff assembly to monitor an oxygen saturation level of the user.
